(19) United States
(12) Patent Application Publication
(10) Pub. No.: US 2014/0350373 A1
(43) Pub. Date: Nov. 27, 2014

(54) OPHTHALMIC LENS WITH A PASSIVE EVENT-BASED COLORATION SYSTEM

(71) Applicant: Johnson & Johnson Vision Care, Inc., Jacksonville, FL (US)

(72) Inventors: Randall B. Pugh, Jacksonville, FL (US); Karson S. Putt, Jacksonville, FL (US); Camille Higham, Jacksonville, FL (US); Sharika Snook, St. Augustine, FL (US)

(73) Assignee: Johnson & Johnson Vision Care, Inc., Jacksonville, FL (US)

(21) Appl. No.: 13/899,516

(22) Filed: May 21, 2013

(51) Int. Cl. A61B 3/10; A61B 5/01

(52) U.S. Cl. CPC A61B 3/101; A61B 5/01
USPC 600/367; 600/549

(57) ABSTRACT

The present invention provides for an Ophthalmic Lens device with passive event coloration mechanisms, which may not require a power source. An Ophthalmic Lens may comprise multiple event coloration mechanisms, wherein the event coloration mechanisms may or may not comprise similar embodiments. The event coloration mechanism may color or change color based on some predefined event. A predefined constituent or predefined condition of the tear fluid may be indicative of the predefined event, and the event coloration mechanisms may interact with the tear fluid, accordingly. In some embodiments, the passive event coloration mechanisms may be combined with Rigid Inserts or Media Inserts, wherein the inserts may provide additional functionalities.

### FIELD OF USE

This invention describes methods, apparatus, and devices related to Ophthalmic Lenses with an event coloration mechanism, wherein the event coloration mechanism may provide a visual indication upon the occurrence of a predefined event. More specifically, the invention describes event coloration mechanisms that may not require a power source.

### BACKGROUND

Traditionally, an Ophthalmic Device, such as a contact lens, an intraocular lens, or a punctal plug included a biocompatible device with a corrective, cosmetic, or therapeutic quality. A contact lens, for example, can provide one or more of vision correcting functionality, cosmetic enhancement, and therapeutic effects. Each function is provided by a physical characteristic of the lens. A design incorporating a refractive quality into a lens can provide a vision corrective function. A pigment incorporated into the lens can provide a cosmetic enhancement. An active agent incorporated into a lens can provide a therapeutic functionality. Such physical characteristics may be accomplished without the lens entering into an energized state.

The functionality of the Ophthalmic Lens may not be limited to ophthalmic functions. When placed on an eye, an Ophthalmic Lens is in contact with the ocular environment, such as, tear fluid, which may include constituents similar to those contained in blood. Accordingly, an Ophthalmic Lens may provide a platform to monitor specific attributes of the ocular environment, such as tear fluid constituents.

It may be desirable to improve the process, methods, and resulting devices for realizing event coloration mechanisms of various kinds. It may be anticipated that some of the solutions for passive event coloration mechanisms inserts may provide novel aspects for energized devices and other biomedical devices. Accordingly, novel methods, devices, and apparatus relating to Ophthalmic Lenses with passive event coloration mechanisms are therefore important.

### SUMMARY

Accordingly, the present invention includes innovations relating to an Ophthalmic Lens with an event coloration mechanism. The Ophthalmic Lens may comprise a soft lens portion, wherein the soft lens portion may comprise a polymerized Reactive Monomer Mixture; and an event coloration mechanism, wherein the event coloration mechanism is capable of triggering a visual indication in the Ophthalmic Lens based on an occurrence of a predefined event, and wherein the event coloration mechanism may be in contact with a portion of the soft lens portion. The event coloration mechanisms may be at least partially visible by the wearer.

In some embodiments, a predefined constituent of the tear fluid may be indicative of the predefined event. For example, the predefined constituent may comprise a pathogen, a biomarker, or an active agent. In some such embodiments, the event coloration mechanism may further comprise a reservoir containing a substance, wherein the substance may be capable of reacting with the predefined constituent, and wherein the reacting is capable of causing a coloration or a color change within the reservoir; and a first encapsulating layer capable of containing the reservoir, wherein the encapsulating layer may be permeable to the predefined constituent. The coloration or color change may be reversible.

Alternatively, a condition within the ocular environment may be indicative of the predefined event. In some such embodiments, the event coloration mechanism may further comprise a reservoir containing a substance, wherein the substance is capable of reacting to the condition, and wherein the reacting is capable of causing coloration or a color change within the reservoir; and an encapsulating layer capable of containing the reservoir. For example, the condition may comprise a temperature within the ocular environment, and the substance may comprise liquid crystal. The liquid crystal may be capable of altering color based on a predefined temperature change in the ocular environment. The coloration or color change may be reversible.

In other alternative examples, the event coloration mechanism may comprise a reactive molecule, wherein the reactive molecule may be capable of reacting with the predefined constituent, and wherein the reactive is capable of causing a coloration or color change in the reactive molecule; and an anchor in proximity to the reactive molecule, wherein the anchor may be capable of securing the reactive molecule within the soft lens portion. The coloration or color change may be reversible.

The reactive molecule may comprise a binding portion capable of binding with the predefined constituent, wherein the binding may be capable of altering a configuration of the reactive molecule; a quenching portion located on a first end of the first binding portion; and a coloration portion located on a second end of the first binding portion. For example, the coloration portion may comprise a fluorophore or a chromophore. The change in configuration may be capable of providing the coloration or color change

Alternatively, the reactive molecule may comprise a binding portion capable of binding with the predefined constituent, wherein the binding is capable of altering a configuration of the reactive molecule; and a Forster resonance energy transfer pair comprising a donor coloration portion located on a first end of the second binding portion; and an acceptor coloration portion located on a second end of the second binding portion capable of accepting energy from the donor coloration portion. The change in configuration may be capable of providing the coloration or color change

In some such embodiments, the anchor may comprise a Rigid Insert. The Rigid Insert may comprise an annular shape capable of framing an Optic Zone of the eye, wherein the Rigid Insert is capable of anchoring a plurality of reactive molecules. The plurality of reactive molecules may be capable of reacting with a plurality of predefined constituents or may be capable of reacting with the same predefined constituent.

In still other embodiments, the event coloration mechanism may further comprise a reservoir comprising a colored substance, wherein the colored substance is limitedly visible when located within the reservoir; and an encapsulating layer capable of encapsulating the reservoir. The occurrence of the predefined event may be capable of degrading the encapsulating layer, wherein the degradation of the third encapsulating layer may be capable of releasing the colored substance from the reservoir. The event coloration mechanism further may further comprise another reservoir in proximity to the first reservoir, wherein the colored substance may be released into the second reservoir, wherein the visibility of the colored substance may be higher in the second reservoir than in the first reservoir.

### DESCRIPTION OF THE DRAWINGS

FIG. 1 illustrates an exemplary embodiment of an Ophthalmic Lens with a passive event coloration mechanism.

FIG. 2 illustrates an alternate embodiment of passive event coloration mechanisms included in an Ophthalmic Lens.

FIG. 3 illustrates an alternate embodiment of passive event coloration mechanisms included in an Ophthalmic Lens.

FIG. 4 illustrates an alternate embodiment of an Ophthalmic Lens with a passive event coloration mechanism.

### DETAILED DESCRIPTION OF THE INVENTION

The present invention describes an Ophthalmic Lens device with passive event coloration mechanisms. In general, according to some embodiments of the present invention, passive event coloration mechanisms may be incorporated with an Ophthalmic Lens, including those with a Rigid Insert or Media Insert. Passive event coloration mechanisms may be “activated” without requiring a power source, but instead may interface or interact with an ocular environment. This proximity with the ocular environment may allow for a wide range of events.

In some embodiments, for example, the event may be a specific or threshold concentration of a biomarker within the tear fluid. Monitoring the concentration of certain biomarkers within tear fluid may allow a patient or doctor to develop a more effective therapy schedule, such as with light therapy and melatonin levels. Alternatively, the coloration may be able to alert the patient of ineffective or dangerous levels of the biomarker, which may be high levels or low levels, depending on the biomarker. For example, high levels of glucose in a diabetic patient may require an emergency response.

An alternative example of an event may be medication levels in the tear fluid. Some medications are most effective within a specific concentration range, and some may even be dangerous at concentrations above that range. Such medications may include, for example, those that treat mental disorders, thyroid diseases, or degenerative brain diseases, such as, Alzheimer's disease.

For example, valproic acid is a common medication that may treat epilepsy or bipolar, in lower doses. Frequent blood tests may be required to monitor the concentration of the medication to ensure the concentration is within the therapeutic range and not into the toxic range, which may cause, for example, renal failure or increase in symptoms of the mental disorder. A constant monitoring system may allow a patient to maintain safe and effective levels.

Other events may include the presence or concentration of specific pathogens, for example, those that may cause ocular infections or may be indicative of non-ocular infections or diseases, such as keratitis, conjunctivitis, corneal ulcers, and cellulitis. Such pathogens may include, for example, Acanthamoeba keratitis, Pseudomona aeruginosa, Neisseria gonorrhoeae, and Staphylococcus and Streptococcus strains, such as S. aureus.

In the following sections, detailed descriptions of embodiments of the invention will be given. The description of both preferred and alternative embodiments are exemplary embodiments only, and it is understood that to those skilled in the art that variations, modifications, and alterations may be apparent. It is therefore to be understood that said exemplary embodiments do not limit the scope of the underlying invention.

### GLOSSARY

In this description and claims directed to the presented invention, various terms may be used for which the following definitions will apply:

Back Curve Piece or Back Insert Piece: as used herein refers to a solid element of a multi-piece Rigid Insert which when assembled into the said insert will occupy a location on the side of the lens that is on the back. In an Ophthalmic Device, said piece would be located on the side of the insert that would be closer to the user's eye surface. In some embodiments, the back curve piece may contain and include a region in the center of an Ophthalmic Device through which light may proceed into the user's eye, which may be called an Optic Zone. In other embodiments, the piece may take an annular shape where it does not contain or include some or all of the regions in an optic zone. In some embodiments of an ophthalmic insert, there may be multiple back curve pieces and one of them may include the optic zone, while others may be annular or portions of an annulus.

Component: as used herein refers to a device capable of drawing electrical current from an Energy Source to perform one or more of a change of logical state or physical state.

Encapsulate: as used herein refers to creating a barrier to separate an entity, such as, for example, a Media Insert, from an environment adjacent to the entity.

Encapsulant: as used herein refers to a layer formed surrounding an entity, such as, for example, a Media Insert, that creates a barrier to separate the entity from an environment adjacent to the entity. For example, Encapsulants may be comprised of silicone hydrogels, such as Etafilcon, Galyfilcon, Narafilcon, and Senofilcon, or other hydrogel contact lens material. In some embodiments, an Encapsulant may be semipermeable to contain specified substances within the entity and preventing specified substances, such as, for example, water, from entering the entity.

Energized: as used herein refers to the state of being able to supply electrical current to or to have electrical energy stored within.

Energy: as used herein refers to the capacity of a physical system to do work. Many uses within this invention may relate to the said capacity being able to perform electrical actions in doing work.

Energy Source: as used herein refers to device capable of supplying Energy or placing a biomedical device in an Energized state.

Event: as used herein refers to a defined set of parameters, such as, for example, a biomarker level, energization level, pH level, or a visual recognition of a particular object. An event may be specific to a wearer, such as a level of medication, or may be generally applicable to all wearers, such as temperature.

Front Curve Piece or Front Insert Piece: as used herein refers to a solid element of a multi-piece Rigid Insert which when assembled into the said insert will occupy a location on the side of the lens that is on the front. In an Ophthalmic Device, a Front Curve Piece would be located on the side of the insert that would be further from the user's eye surface. In some embodiments, the piece may contain and include a region in the center of an Ophthalmic Device through which light may proceed into the user's eye, which may be called an Optic Zone. In other embodiments, the piece may take an annular shape where it does not contain or include some or all of the regions in an optic zone. In some embodiments of an ophthalmic insert, there may be multiple front curve pieces and one of them may include the optic zone, while others may be annular or portions of an annulus.

Lens-forming mixture or Reactive Mixture or Reactive Monomer Mixture (RMM): as used herein refers to a monomer or prepolymer material that can be cured and cross-linked or cross-linked to form an Ophthalmic Lens. Various embodiments can include lens-forming mixtures with one or more additives such as UV blockers, tints, photoinitiators or catalysts, and other additives one might desire in an Ophthalmic Lenses such as, contact or intraocular lenses.

Lens-forming Surface: as used herein refers to a surface that is used to mold a lens. In some embodiments, any such surface can have an optical quality surface finish, which indicates that it is sufficiently smooth and formed so that a lens surface fashioned by the polymerization of a lens forming material in contact with the molding surface is optically acceptable. Further, in some embodiments, the lens-forming surface can have a geometry that is necessary to impart to the lens surface the desired optical characteristics, including without limitation, spherical, aspherical and cylinder power, wave front aberration correction, corneal topography correction and the like as well as any combinations thereof.

Liquid Crystal: as used herein refers to a state of matter having properties between a conventional liquid and a solid crystal. A Liquid Crystal cannot be characterized as a solid but its molecules exhibit some degree of alignment. As used herein, a Liquid Crystal is not limited to a particular phase or structure, but a Liquid Crystal may have a specific resting orientation. The orientation and phases of a Liquid Crystal may be manipulated by external forces such as, for example, temperature, magnetism, or electricity, depending on the class of Liquid Crystal.

Lithium Ion Cell: as used herein refers to an electrochemical cell where Lithium ions move through the cell to generate electrical energy. This electrochemical cell, typically called a battery, may be reenergized or recharged in its typical forms.

Media Insert: as used herein refers to an encapsulated insert that will be included in an energized Ophthalmic Device. The energization elements and circuitry may be embedded in the Media Insert. The Media Insert defines the primary purpose of the energized Ophthalmic Device. For example, in embodiments where the energized Ophthalmic Device allows the user to adjust the optic power, the Media Insert may include energization elements that control a liquid meniscus portion in the Optical Zone. Alternatively, a Media Insert may be annular so that the Optical Zone is void of material. In such embodiments, the energized function of the Lens may not be optic quality but may be, for example, monitoring glucose or administering medicine.

Mold: as used herein refers to a rigid or semi-rigid object that may be used to form lenses from uncured formulations. Some preferred molds include two mold parts forming a front curve Mold part and a back curve Mold part.

Ophthalmic Lens or Ophthalmic Device or Lens: as used herein refers to any device that resides in or on the eye, in contrast to a spectacle lens. The device may provide optical correction, may be cosmetic, or provide some functionality unrelated to optic quality. For example, the term Lens may refer to a contact Lens, intraocular Lens, overlay Lens, ocular insert, optical insert, or other similar device through which vision is corrected or modified, or through which eye physiology is cosmetically enhanced (e.g. iris color) without impeding vision. Alternatively, Lens may refer to a device that may be placed on the eye with a function other than vision correction, such as, for example, monitoring of a constituent of tear fluid or means of administering an active agent. In some embodiments, the preferred Lenses of the invention may be soft contact Lenses that are made from silicone elastomers or hydrogels, which may include, for example, silicone hydrogels and fluorohydrogels.

Optic Zone: as used herein refers to an area of an Ophthalmic Lens through which a wearer of the Ophthalmic Lens sees.

Power: as used herein refers to work done or energy transferred per unit of time.

Reenergize or Recharge: as used herein refers to a restoration to a state with higher capacity to do work. Many uses within this invention may relate to restoring a device to the capability to flow electrical current at a certain rate for a specified, reestablished time period.

Released from a mold: as used herein refers to a lens is either completely separated from the mold, or is only loosely attached so that it can be removed with mild agitation or pushed off with a swab.

Rigid Insert: as used herein refers to an insert that maintains a predefined topography and includes a greater modulus than a hydrogel in contact with all or part of the Rigid Insert. When included in a Contact Lens, the Rigid Insert may contribute to the functionality of the Lens. For example, varying topography of or densities within the Rigid Insert may define zones, which may correct vision in users with astigmatism.

Three-dimensional Surface or Three-dimensional Substrate: as used herein refers to any surface or substrate that has been three-dimensionally formed where the topography is designed for a specific purpose, in contrast to a planar surface.

Ophthalmic Lens with an Event Coloration Mechanism

### Non-Energizable Event Coloration Mechanisms

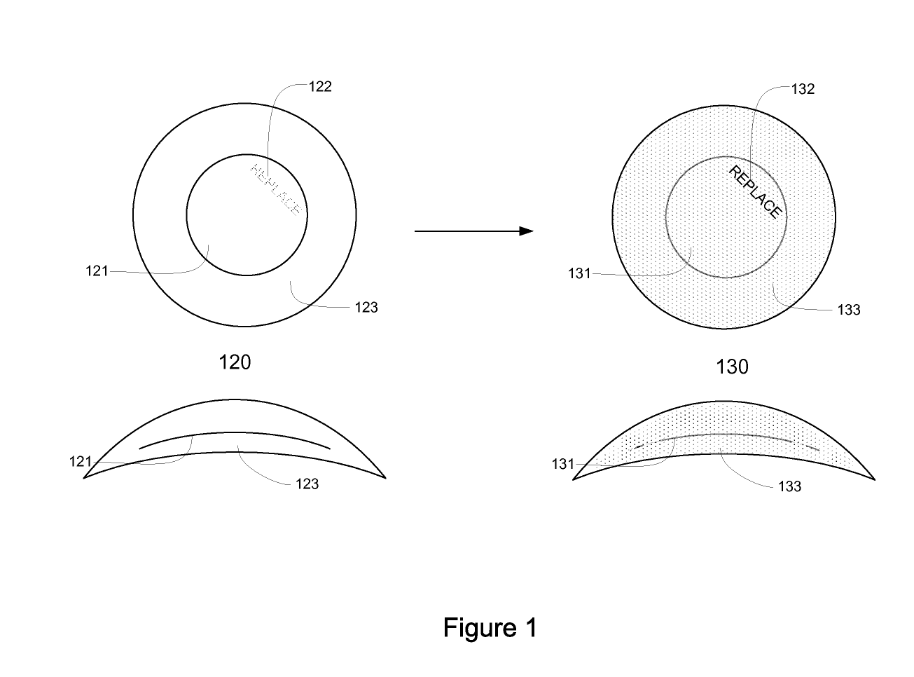

Proceeding to FIG. 1, an example of a Rigid Insert 121 with a non-energizable event coloration mechanism 122 is illustrated. In some embodiments, an event coloration mechanism 122 may comprise a reactive mixture, which, for example, may be added to, printed on, or embedded in a Rigid Insert 121, such as through thermoforming techniques. Alternatively, not shown, the event coloration mechanism 122 may not require a Rigid Insert 121 but instead may be located on or within the hydrogel portion 123, for example, through use of printing or injection techniques.

The event coloration mechanism 122 may comprise a portion of the Rigid Insert 121 that is reactive to some component of the transient tear fluid or some component within the Ophthalmic Lens 120. For example, the event may be a specific accumulation of some precipitant, such as, lipids or proteins, on either or both the Rigid Insert 131 and the hydrogel portion 133, depending on the composition of the Ophthalmic Lens 130. The accumulation level may “activate” the event coloration mechanism 132 without requiring a power source. The activation may be gradual wherein the color becomes more visible as the accumulation level increases, which may indicate when the Ophthalmic Lens 130 needs to be cleaned or replaced.

Alternatively, the color may only be apparent at a specific level. In some embodiments, the activation may be reversible, such as, for example, where the wearer effectively removes the precipitant from the hydrogel portion 133 or the Rigid Insert 131. The event coloration mechanism 132 may be located outside the Optic Zone, which may allow for an annular embodiment of the Rigid Insert, not shown. In other embodiments, particularly where the event may prompt a wearer to take immediate action, the event coloration mechanism 132 may be located within the Optic Zone, allowing the wearer to see the activation of the event coloration mechanism 132.

In some other embodiments, the event coloration mechanism, not shown, may comprise a reservoir containing a colored substance, such as, for example, a dye. Prior to the occurrence of the event, the reservoir may not be visible. The reservoir may be encapsulated with a degradable material, which may be irreversibly degraded by some constituent of the tear fluid, including, for example, proteins or lipids. Once degraded, the colored substance may be released into the Ophthalmic Lens 130 or into a second reservoir. Such an embodiment may indicate when a disposable Ophthalmic Lens 130 should be disposed, for example, based on a manufacturer's recommended parameters.

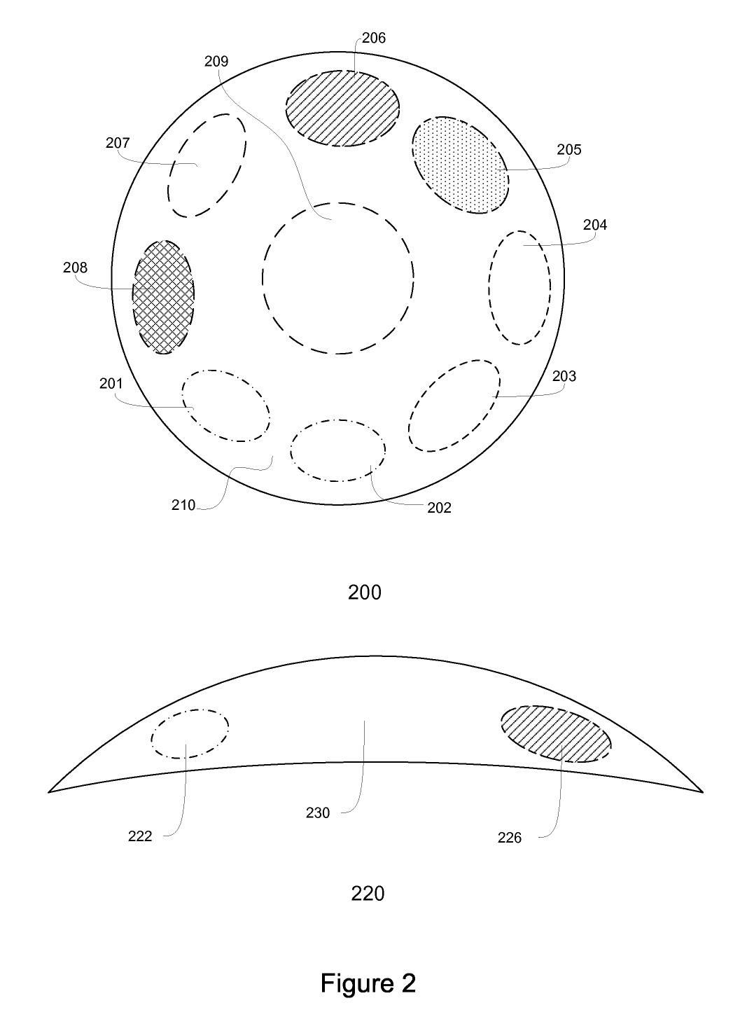

Proceeding to FIG. 2, an exemplary embodiment of an Ophthalmic Lens 200 with multiple event coloration mechanisms 201-208 is illustrated. In some embodiments, the event coloration mechanisms 201-208 may be located within the soft, hydrogel portion 210 of the Ophthalmic Lens 200 and outside the Optic Zone 209. Such embodiments may not require a Rigid Insert or Media Insert for functioning of the event coloration mechanisms 201-208, though inserts may still be incorporated in the Ophthalmic Lens 200 allowing for additional functionalities.

In some embodiments, each event coloration mechanism 201-208 may be separately encapsulated within the soft, hydrogel portion 210 of the Ophthalmic Lens. The contents of the event coloration mechanisms 201-208 may include a compound reactive to some condition, such as temperature, or component of tear fluid, such as a biomarker.

In some embodiments, each event coloration mechanism 201-208 may “activate” based on different events. For example, one event coloration mechanism 208 may comprise liquid crystal that may react to changes in temperatures of the ocular environment, wherein the event is a fever. Other event coloration mechanisms 202-206 within the same Ophthalmic Lens 200 may react to specific pathogens, for example, those that may cause ocular infections or may be indicative of non-ocular infections or diseases, such as keratitis, conjunctivitis, corneal ulcers, and cellulitis. Such pathogens may include, for example, Acanthamoeba keratitis, Pseudomona aeruginosa, Neisseria gonorrhoeae, and Staphylococcus and Streptococcus strains, such as S. aureus.

The event coloration mechanisms 201-207 may be encapsulated with a compound that may be selectively permeable to a component of tear fluid, such as, for example, through use of a Nafion lining. In some embodiments, the event coloration mechanism 202-206 may function by agglutination, such as through a coagulase test, wherein a higher concentration of the pathogen may adhere to a compound within the event coloration mechanism 202-206 and may cause clumping or the formation of precipitate. The precipitate may provide coloration or may react with another compound in the event coloration mechanism 202-206 through a separate reaction. Alternatively, the event coloration mechanism 202-206 may comprise a reagent that colors upon reaction, such as with some oxidase tests.

In still other embodiments, an event coloration mechanism 202-206 may function similarly to a litmus test, wherein the event coloration mechanism activates based on the pH or pOH within the ocular environment. For example, to monitor the concentration of valproic acid, the event coloration mechanism may contain specific proteins that would be able to bind to the valproic acid up to a specific concentration. The non-binding valproic acid may be indicative of the effective quantities within the tear fluid. The pH or pOH within the event coloration mechanism may increase with the increased concentration of the acid.

Other exemplary coloration mechanisms 201 may be reactive to ultraviolet rays, wherein the event may be overexposure of the eye to UV light, as with snow blindness. Another coloration mechanism 207 may react to protein accumulation, such as described with FIG. 1.

Some event coloration mechanisms 208 may be reversible, such as when the wearer has effectively responded to the event. For example, after a wearer has rinsed the Ophthalmic Lens, the level of pathogens or protein may be sufficiently reduced to allow for safe use of the Ophthalmic Lens. Alternatively, the coloration may be reversible on the eye, such as where the event is a fever and the wearer's temperature has been effectively lowered.

As shown in cross section, the event coloration mechanisms 222, 226 may be located in the periphery of the Ophthalmic Lens 220 without altering the optical surface of the hydrogel portion 230. In some embodiments, not shown, the event coloration mechanisms may be at least partially within the Optic Zone 229, alerting the wearer of the event. The locations of the event coloration mechanisms 222, 226 may be varied within a single Ophthalmic Lens 220, with some in the periphery and some within the Optic Zone 229.

The event coloration mechanisms 201-208 may be independently activated. For example, the wearer may have a fever, triggering a change in coloration in liquid crystal contained in an event coloration mechanism 208. Two other event coloration mechanisms 205, 206 may indicate high levels of S. aureus and A. keratitis, which may provide guidance on what is causing the fever, particularly where other symptoms corroborate the diagnosis. Where the event coloration mechanisms 201-208 serve as diagnostic tools, the coloration may not be reversible, allowing the wearer to remove the Ophthalmic Lens 200 without losing the event indication.

In some embodiments, the event coloration mechanism 208 may be coated in a substance with low permeability, such as, for example, parylene. This embodiment may be particularly significant where the event coloration mechanism 208 contains compounds that may be dangerous if in contact with the eye or where the event does not require interaction with the tear fluid. For example, where the event is a temperature change, a liquid crystal droplet may be parylene coated, which may be further strengthened into a hermetic seal by alternating the parylene with a fortifying compound, such as, silicon dioxide, gold, or aluminum.

For exemplary purposes, the Ophthalmic Lens 200 is shown to include eight event coloration mechanisms. However, it may be obvious to those skilled in the art that other quantities of event coloration mechanisms may be practical.

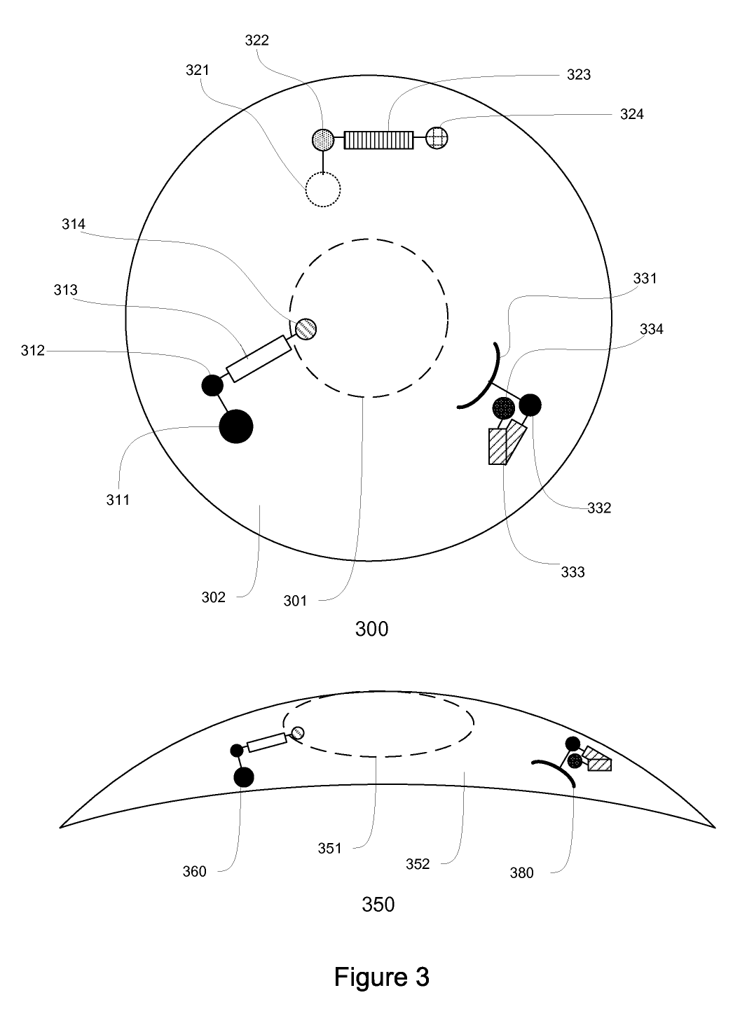

Proceeding to FIG. 3, an alternative embodiment of an Ophthalmic Lens 300 with event coloration mechanisms 311-314, 321-324, 331-334 is illustrated. In some such embodiments, the event mechanisms 311-314, 321-324, 331-334 may include a reactive molecule 312-314, 322-324, 332-334 anchored within the Ophthalmic Lens.

The reactive molecule 312-314, 332-334 may comprise a central binding portion 313, 333 flanked by a quencher 312, 331 and a coloration portion 314, 334, such as, for example, a chromophore or fluorophore. Depending on the molecular structure, when a specified compound binds to the binding portion 313, 333, the coloration portion 314, 334 may shift closer to the quencher 312, reducing coloration, or may shift away from the quencher 332, which would increase coloration. In other embodiments, the reactive molecule 322-324 may comprise a binding portion 323 flanked by Förster resonance energy transfer (FRET) pairs 322, 324. FRET pairs 322, 324 may function similarly to a quencher 312, 332 and chromophore 314, 334, though FRET pairs 322, 324 may both exhibit coloration and, when in close proximity to each other, their spectral overlap may cause a change in coloration.

The reactive molecule 312-314, 322-324, 332-334 may be selected to target specific compounds within the tear fluid. In some embodiments, the specific compound may directly indicate the event. For example, where a level of glucose in the tear fluid is the event, the reactive molecule 312-314, 322-324, 332-334 may directly bind with the glucose. Where the event is the presence or concentration of a pathogen, for example, a particular aspect of that pathogen may bind with the reactive molecule 312-314, 322-324, 332-334. This may include a unique lipid or protein component of that pathogen. Alternatively, the specific compound may be an indirect indicator of the event. The specific compound may be a byproduct of the pathogen, such as a particular antibody that responds to that pathogen.

In some embodiments, the reactive molecule 312-314 may be anchored within the Ophthalmic Lens by a secondary compound 311, such as, for example, a protein, peptide, or aptamer. Alternatively, the hydrogel 302 may provide a sufficient anchor 321 to secure the reactive molecule 322-324 within the Ophthalmic Lens 300. The reactive molecule 322-324 may be in contact with the Reactive Monomer Mix prior to polymerization, which may allow the reactive molecule 322-324 to chemically bind with the hydrogel 321. The reactive molecule may be injected into the hydrogel after polymerization but before hydration, which may allow precise placement of the reactive molecule.

In some embodiments, tinting the anchoring mechanism may provide broader cosmetic choices. The Ophthalmic Lens may further comprise a limbic ring or an iris pattern, which may provide a static and natural background or foreground to the event coloration mechanisms. The design pattern may be included on or within the hydrogel or may be included in a Rigid Insert through a variety of processes, such as, for example, printing on a surface of the Rigid Insert. In some such embodiments, the periphery event coloration mechanisms may be arranged to appear less artificial, for example through a sunburst pattern that may more naturally integrate into the wearer's iris pattern or an iris pattern included in the Ophthalmic Lens than random dotting throughout the Ophthalmic Lens.

In other embodiments, the reactive molecule 332-334 may be anchored to a Rigid Insert 331. The Rigid Insert, not shown, may be annular and may anchor multiple reactive molecules outside of the Optic Zone 301. Alternatively, the Rigid Insert 331 may be a small periphery insert, which may anchor a single reactive molecule 332-334 or many of the same reactive molecules, which may allow for a more vibrant coloration.

As illustrated in cross section, the placement of the reactive molecules 360, 380 within the Ophthalmic Lens 350 may be varied within the hydrogel 352. For example, some reactive molecules 380 may be entirely in the periphery with no overlap with the Optic Zone 351. Other reactive molecules 360 may at least partially extend into the Optic Zone 351. In some such embodiments, the reactive molecules 360 may extend into the Optic Zone 351 in some configurations of that reactive molecule 360, such as when the event has occurred, which may alert the wearer of the event.

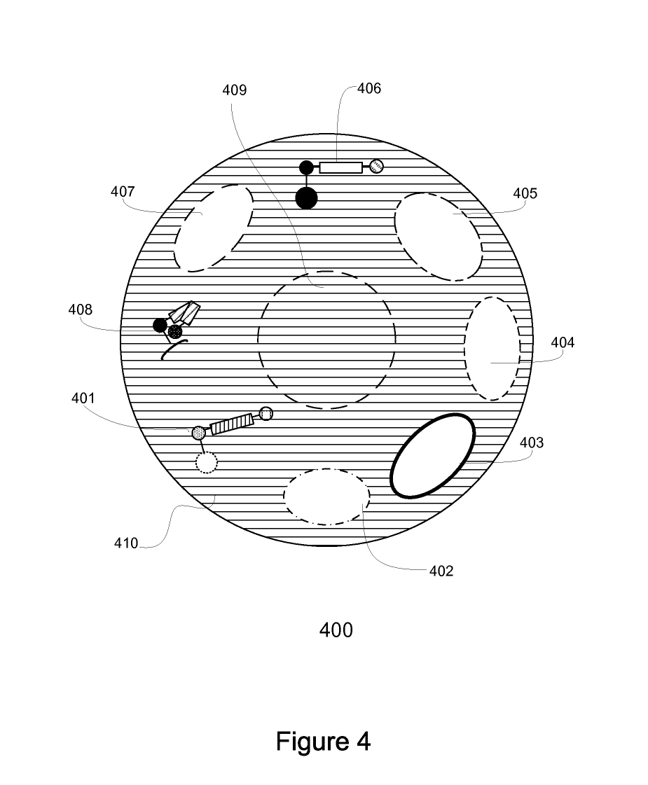

Proceeding to FIG. 4, an exemplary embodiment of an Ophthalmic Lens 400 with a variety of event coloration mechanisms 401-408, 410 is illustrated. Some Ophthalmic Lenses 400 may comprise a combination of event coloration mechanism embodiments. For example, the event coloration mechanisms may include FRET pairs 401, parylene-coated liquid crystal 403, Rigid Insert anchored quencher 408, secondary compound anchored quencher 406, selectively permeable reservoirs 402, 404, 405, 407.

An event coloration mechanism may also be integrated with the hydrogel portion 410. For example, a reactive molecule may be mixed with the Reactive Monomer Mixture prior to polymerization. In some such embodiments, the event coloration mechanism may be dispersed throughout the hydrogel 410, including over the Optic Zone 409.

In some embodiments, the varied event coloration mechanisms 401-408 may indicate a mix of events, which may be tailored according to a patient's needs. For example, the event coloration mechanisms may indicate a series of event related to a single disorder. In some such embodiments, one event coloration mechanism may change color with an increase in glucose levels, and another event coloration mechanism may lose color when a diabetic medication is in low concentration. Such an embodiment may act as a reminder to the patient to take their medication or may allow the patient to eat accordingly. Another example may include monitoring serotonin levels in a first event coloration mechanism and depression medications in other event coloration mechanisms. This may be particularly significant where a patient takes a combination of medications.

Materials for Insert Based Ophthalmic Lenses

In some embodiments, a lens type can be a lens that includes a silicone-containing component. A “silicone-containing component” is one that contains at least one [—Si—O—] unit in a monomer, macromer or prepolymer. Preferably, the total Si and attached 0 are present in the silicone-containing component in an amount greater than about 20 weight percent, and more preferably greater than 30 weight percent of the total molecular weight of the silicone-containing component. Useful silicone-containing components preferably comprise polymerizable functional groups such as acrylate, methacrylate, acrylamide, methacrylamide, vinyl, N-vinyl lactam, N-vinylamide, and styryl functional groups.

In some embodiments, the Ophthalmic Lens skirt, which sometimes may be called an insert encapsulating layer, that surrounds the insert may be comprised of standard hydrogel lens formulations. Exemplary materials with characteristics that may provide an acceptable match to numerous insert materials may include the Narafilcon family; including Narafilcon A and Narafilcon B. Alternatively, the Etafilcon family; including Etafilcon A may represent good exemplary material choices. A more technically inclusive discussion follows on the nature of materials consistent with the art herein; but it may be clear that any material that may form an acceptable enclosure or partial enclosure of the sealed and encapsulated inserts are consistent and included.

Suitable silicone containing components include compounds of Formula I

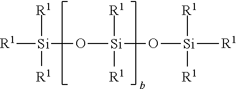

where:

R¹ is independently selected from monovalent reactive groups, monovalent alkyl groups, or monovalent aryl groups, any of the foregoing which may further comprise functionality selected from hydroxy, amino, oxa, carboxy, alkyl carboxy, alkoxy, amido, carbamate, carbonate, halogen or combinations thereof; and monovalent siloxane chains comprising 1-100 Si—O repeat units which may further comprise functionality selected from alkyl, hydroxy, amino, oxa, carboxy, alkyl carboxy, alkoxy, amido, carbamate, halogen or combinations thereof;

where b=0 to 500, where it is understood that when b is other than 0, b is a distribution having a mode equal to a stated value;

wherein at least one R¹ comprises a monovalent reactive group, and in some embodiments between one and 3 R¹ comprise monovalent reactive groups.

As used herein “monovalent reactive groups” are groups that can undergo free radical and/or cationic polymerization. Non-limiting examples of free radical reactive groups include (meth)acrylates, styryls, vinyls, vinyl ethers, C₁₋₆alkyl(meth)acrylates, (meth)acrylamides, C₁₋₆alkyl(meth)acrylamides, N-vinyllactams, N-vinylamides, C₂₋₁₂ alkenyls, C₂₋₁₂alkenylphenyls, C₂₋₁₂alkenylnaphthyls, C₂₋₆alkenylphenylC₁₋₆alkyls, O-vinylcarbamates and O-vinylcarbonates. Non-limiting examples of cationic reactive groups include vinyl ethers or epoxide groups and mixtures thereof. In one embodiment the free radical reactive groups comprises (meth)acrylate, acryloxy, (meth)acrylamide, and mixtures thereof.

Suitable monovalent alkyl and aryl groups include unsubstituted monovalent C₁ to C₁₆alkyl groups, C₆-C₁₄ aryl groups, such as substituted and unsubstituted methyl, ethyl, propyl, butyl, 2-hydroxypropyl, propoxypropyl, polyethyleneoxypropyl, combinations thereof and the like.

In one embodiment b is zero, one R¹ is a monovalent reactive group, and at least 3 R¹ are selected from monovalent alkyl groups having one to 16 carbon atoms, and in another embodiment from monovalent alkyl groups having one to 6 carbon atoms. Non-limiting examples of silicone components of this embodiment include 2-methyl-,2-hydroxy-3-[3-[1,3,3,3-tetramethyl-1-[(trimethylsilyl)oxy]disiloxanyl]propoxy]propyl ester (“SiGMA”), 2-hydroxy-3-methacryloxypropyloxypropyl-tris(trimethylsiloxy)silane, 3-methacryloxypropyltris(trimethylsiloxy)silane (“TRIS”), 3-methacryloxypropylbis(trimethylsiloxy)methylsilane and 3-methacryloxypropylpentamethyl disiloxane.

In another embodiment, b is 2 to 20, 3 to 15 or in some embodiments 3 to 10; at least one terminal R¹ comprises a monovalent reactive group and the remaining R¹ are selected from monovalent alkyl groups having 1 to 16 carbon atoms, and in another embodiment from monovalent alkyl groups having 1 to 6 carbon atoms. In yet another embodiment, b is 3 to 15, one terminal R¹ comprises a monovalent reactive group, the other terminal R¹ comprises a monovalent alkyl group having 1 to 6 carbon atoms and the remaining R¹ comprise monovalent alkyl group having 1 to 3 carbon atoms. Non-limiting examples of silicone components of this embodiment include (mono-(2-hydroxy-3-methacryloxypropyl)-propyl ether terminated polydimethylsiloxane (400-1000 MW)) (“OH-mPDMS”), monomethacryloxypropyl terminated mono-n-butyl terminated polydimethylsiloxanes (800-1000 MW), (“mPDMS”).

In another embodiment b is 5 to 400 or from 10 to 300, both terminal R¹ comprise monovalent reactive groups and the remaining R¹ are independently selected from monovalent alkyl groups having 1 to 18 carbon atoms which may have ether linkages between carbon atoms and may further comprise halogen.

In one embodiment, where a silicone hydrogel lens is desired, the lens of the present invention will be made from a Reactive Mixture comprising at least about 20 and preferably between about 20 and 70% wt silicone containing components based on total weight of reactive monomer components from which the polymer is made.

In another embodiment, one to four R¹ comprises a vinyl carbonate or carbamate of the formula:

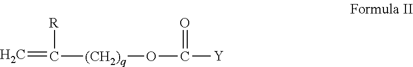

wherein: Y denotes O—, S— or NH—;

R denotes, hydrogen or methyl; d is 1, 2, 3 or 4; and q is 0 or 1.

The silicone-containing vinyl carbonate or vinyl carbamate monomers specifically include: 1,3-bis[4-(vinyloxycarbonyloxy)but-1-yl]tetramethyl-disiloxane; 3-(vinyloxycarbonylthio) propyl-[tris(trimethylsiloxy)silane]; 3-[tris(trimethylsiloxy)silyl]propyl allyl carbamate; 3-[tris(trimethylsiloxy)silyl]propyl vinyl carbamate; trimethylsilylethyl vinyl carbonate; trimethylsilylmethyl vinyl carbonate, and

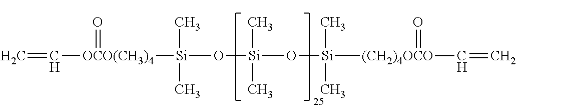

Where biomedical devices with modulus below about 200 are desired, only one R¹ shall comprise a monovalent reactive group and no more than two of the remaining R¹ groups will comprise monovalent siloxane groups.

Another class of silicone-containing components includes polyurethane macromers of the following formulae:

(*D*A*D*G)ₐ*D*D*E¹;

E(*D*G*D*A)ₐ*D*G*D*E¹ or;

E(*D*A*D*G)ₐ*D*A*D*E¹ Formulae IV-VI

wherein:

D denotes an alkyl diradical, an alkyl cycloalkyl diradical, a cycloalkyl diradical, an aryl diradical or an alkylaryl diradical having 6 to 30 carbon atoms,

G denotes an alkyl diradical, a cycloalkyl diradical, an alkyl cycloalkyl diradical, an aryl diradical or an alkylaryl diradical having 1 to 40 carbon atoms and which may contain ether, thio or amine linkages in the main chain;

* denotes a urethane or ureido linkage;

ₐ is at least 1;

A denotes a divalent polymeric radical of formula:

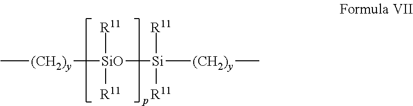

R¹¹ independently denotes an alkyl or fluoro-substituted alkyl group having 1 to10 carbon atoms which may contain ether linkages between carbon atoms; y is at least 1; and p provides a moiety weight of 400 to 10,000; each of E and E¹ independently denotes a polymerizable unsaturated organic radical represented by formula:

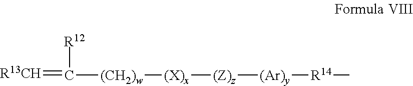

wherein: R¹² is hydrogen or methyl; R¹³ is hydrogen, an alkyl radical having 1 to 6 carbon atoms, or a —CO—Y—R¹⁵ radical wherein Y is —O—, Y—S— or —NH—; R¹⁴ is a divalent radical having 1 to 12 carbon atoms; X denotes —CO— or —OCO—; Z denotes —O— or —NH—; Ar denotes an aromatic radical having 6 to 30 carbon atoms; w is 0 to 6; x is 0 or 1; y is 0 or 1; and z is 0 or 1.

A preferred silicone-containing component is a polyurethane macromer represented by the following formula:

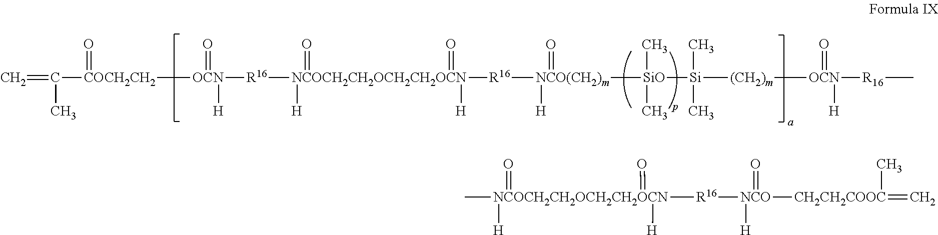

wherein R¹⁶ is a diradical of a diisocyanate after removal of the isocyanate group, such as the diradical of isophorone diisocyanate. Another suitable silicone containing macromer is compound of formula X (in which x+y is a number in the range of 10 to 30) formed by the reaction of fluoroether, hydroxy-terminated polydimethylsiloxane, isophorone diisocyanate and isocyanatoethylmethacrylate.

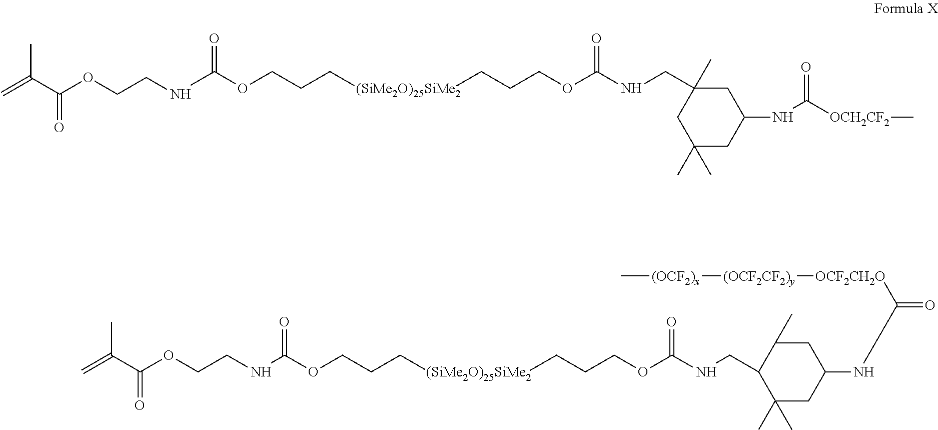

Other silicone containing components suitable for use in this invention include macromers containing polysiloxane, polyalkylene ether, diisocyanate, polyfluorinated hydrocarbon, polyfluorinated ether and polysaccharide groups; polysiloxanes with a polar fluorinated graft or side group having a hydrogen atom attached to a terminal difluoro-substituted carbon atom; hydrophilic siloxanyl methacrylates containing ether and siloxanyl linkanges and crosslinkable monomers containing polyether and polysiloxanyl groups. Any of the foregoing polysiloxanes can also be used as the silicone-containing component in this invention.

### CONCLUSION

The present invention, as described above and as further defined by the claims below, provides an Ophthalmic Lens with passive event coloration mechanisms, which may not require a power source. An Ophthalmic Lens may comprise multiple event coloration mechanisms, wherein the event coloration mechanisms may or may not comprise similar embodiments. The event coloration mechanism may color or change color based on some predefined event. A predefined constituent or predefined condition of the tear fluid may be indicative of the predefined event, and the event coloration mechanisms may interact with the tear fluid, accordingly. In some embodiments, the passive event coloration mechanisms may be combined with Rigid Inserts or Media Inserts, wherein the inserts may provide additional functionalities.

1. An Ophthalmic Lens with an event coloration mechanism, the Ophthalmic Lens comprising:
a soft lens portion, wherein the soft lens portion comprises a polymerized Reactive Monomer Mixture; and
an event coloration mechanism, wherein the event coloration mechanism is capable of triggering a visual indication in the Ophthalmic Lens based on an occurrence of a predefined event, and wherein the event coloration mechanism is in contact with a portion of the soft lens portion.

2. The Ophthalmic Lens of claim 1, wherein a predefined constituent of a tear fluid is indicative of the predefined event, and wherein the event coloration mechanism further comprises:
a first reservoir containing a first substance, wherein the first substance is capable of reacting with the predefined constituent, wherein the reacting is capable of causing a coloration or a color change within the first reservoir; and
a first encapsulating layer capable of containing the first reservoir, wherein the encapsulating layer is permeable to the predefined constituent.

3. The Ophthalmic Lens of claim 1, wherein a condition within an ocular environment is indicative of the predefined event, and wherein the event coloration mechanism further comprises:
a second reservoir containing a second substance, wherein the second substance is capable of reacting to the condition, and wherein the reacting is capable of causing a coloration or a color change within the second reservoir; and
a second encapsulating layer capable of containing the first reservoir.

4. The Ophthalmic Lens of claim 1, wherein the event coloration mechanism comprises
a reactive molecule, wherein the reactive molecule is capable of reacting with the predefined constituent, and wherein the reacting is capable of causing a coloration or color change in the reactive molecule; and
an anchor in proximity to the reactive molecule, wherein the anchor is capable of securing the reactive molecule within the soft lens portion.

5. The Ophthalmic Lens of claim 1, wherein the event coloration mechanism further comprises:
a third reservoir comprising a colored substance, wherein the colored substance is limitedly visible when located within the third reservoir; and
a third encapsulating layer capable of encapsulating the reservoir, wherein the occurrence of the predefined event is capable of degrading the third encapsulating layer, and wherein a degradation of the third encapsulating layer is capable of releasing the colored substance from the third reservoir.

6. The Ophthalmic Lens of claim 1, wherein the event coloration mechanisms is at least partially visible by the wearer.

7. The Ophthalmic Lens of claim 2, wherein the predefined constituent comprises a pathogen.

8. The Ophthalmic Lens of claim 2, wherein the predefined constituent comprises a biomarker.

9. The Ophthalmic Lens of claim 2, the predefined constituent comprises an active agent.

10. The Ophthalmic Lens of claim 2, wherein the coloration or color change is reversible.

11. The Ophthalmic Lens of claim 3, wherein the condition comprises a temperature within the ocular environment, wherein the second substance comprises liquid crystal, and wherein the liquid crystal is capable of altering color based on a predefined temperature change in the ocular environment.

12. The Ophthalmic Lens of claim 3, wherein the coloration or color change is reversible.

13. The Ophthalmic Lens of claim 4, wherein the reactive molecule comprises:
a first binding portion capable of binding with the predefined constituent, and wherein the binding is capable of altering a configuration of the reactive molecule, and wherein a change in configuration is capable of providing the coloration or color change;
a quenching portion located on a first end of the first binding portion; and
a coloration portion located on a second end of the first binding portion comprising.

14. The Ophthalmic Lens of claim 4, wherein the reactive molecule comprises:
a second binding portion capable of binding with the predefined constituent, and wherein the binding is capable of altering a configuration of the reactive molecule, and wherein a change in configuration is capable of providing the coloration or color change; and
a Förster resonance energy transfer pair comprising:
a donor coloration portion located on a first end of the second binding portion; and
an acceptor coloration portion located on a second end of the second binding portion capable of accepting energy from the donor coloration portion.

15. The Ophthalmic Lens of claim 4, wherein the coloration or color change is reversible.

16. The Ophthalmic Lens of claim 4, wherein the anchor comprises a Rigid Insert.

17. The Ophthalmic Lens of claim 5, wherein the event coloration mechanism further comprises a fourth reservoir in proximity to the third reservoir, wherein the degradation is capable of releasing the colored substance into the fourth reservoir, and wherein a visibility of the colored substance is higher when located in the fourth reservoir than when located in the third reservoir.

18. The Ophthalmic Lens of claim 16, wherein the Rigid Insert comprises an annular shape capable of framing an Optic Zone of the eye, and wherein the Rigid Insert is capable of anchoring a plurality of reactive molecules.

19. The Ophthalmic Lens of claim 18, wherein the plurality of reactive molecules are capable of reacting with a plurality of predefined constituents.

20. The Ophthalmic Lens of claim 18, wherein the plurality of reactive molecules are capable of reacting with a same predefined constituent.
